(19) United States
(12) Patent Application Publication
(10) Pub. No.: US 2024/0024280 A1
(43) Pub. Date: Jan. 25, 2024

(54) SENOTHERAPEUTIC SUBSTANCE

(71) Applicant: BIONEXA S.R.L, Bari (BA) (IT)

(72) Inventors: Venera RUSSO, Mascali (CT) (IT); Giovanni Mario PITARI, Augusta (SR) (IT); Claudia Giovanna LEOTTA, Sant'Agata li Battiati (CT) (IT); Mario CORREALE, Bari (BA) (IT); Paolo CORREALE, Milano (IT)

(21) Appl. No.: 18/254,979

(22) PCT Filed: Nov. 30, 2021
(86) PCT No.: PCT/IB2021/061101

§ 371 Date: May 30, 2023

(30) Foreign Application Priority Data
Dec. 1, 2020 (IT) 102020000029213

(51) Int. Cl. A61K 31/353; A61K 31/20; A61K 31/192

(52) U.S. Cl. CPC A61K 31/353; A61K 31/20; A61K 31/192

(57) ABSTRACT

A senotherapeutic substance including flavonoids, fatty acids, and, preferably, phenolic acids and/or vitamins. The substance may be used for the manufacture of a medicament for senotherapeutic use.

The present invention relates to a senotherapeutic substance of the type specified in the preamble of the first claim.

Substances, and in particular foods, for special medical purposes are currently known. These are specially formulated foods intended for the dietary management of a disease that has nutritional needs that are not met by the normal diet alone. Furthermore, the existence of senescent cells and the consequent development of a class of drugs known as senotherapeutics, in particular senolytic, or senostatic or senomorphic, has recently been discovered. For example, senolytics are substances which, when taken by a user, selectively kill senescent cells in the human or animal body.

Senescent cells are cells that are no longer able to divide and multiply. They are also subject to loss of physiological function, resistance to apoptosis and various cellular changes.

In addition, senescent cells contribute to the phenotype of aging, including frailty syndrome, sarcopenia and diseases associated with aging. Senescent astrocytes and microglia contribute to neurodegeneration.

The goal of senotherapeutics, and in particular of senolytics, is therefore to delay, prevent, alleviate or reverse age-related diseases by eliminating, as selectively as possible, senescent cells.

Senolytic compounds have been studied for example by the Mayo Foundation for Medical Education and Research (Minnesota—US) for example in patent applications WO2015116735A1, WO2019183282A1 and US2015296755A1. Other senolytic compounds have been developed by the company Unity Biotechnology (California, US), for example in patent applications WO2019241567A1, US2019330199A1 and CA3043103A1.

However, the demand for senotherapeutics, and in particular for senolytics, more precise, performing or cheaper, is always greater.

In this situation, the technical task underlying the present invention is to devise a senotherapeutic substance, capable of substantially obviating at least part of the aforementioned drawbacks.

Within the scope of said technical task, it is an important object of the invention to obtain a senotherapeutic substance which functions selectively on senescent cells. Another important technical task is to make a senotherapeutic substance whose production is economical.

The technical task and the specified aims are achieved by a senotherapeutic substance as claimed in the attached claim 1.

Examples of preferred embodiment are described in the dependent claims.

The characteristics and advantages of the invention are clarified below by the detailed description of preferred embodiments of the invention, with reference to the accompanying drawings, in which:

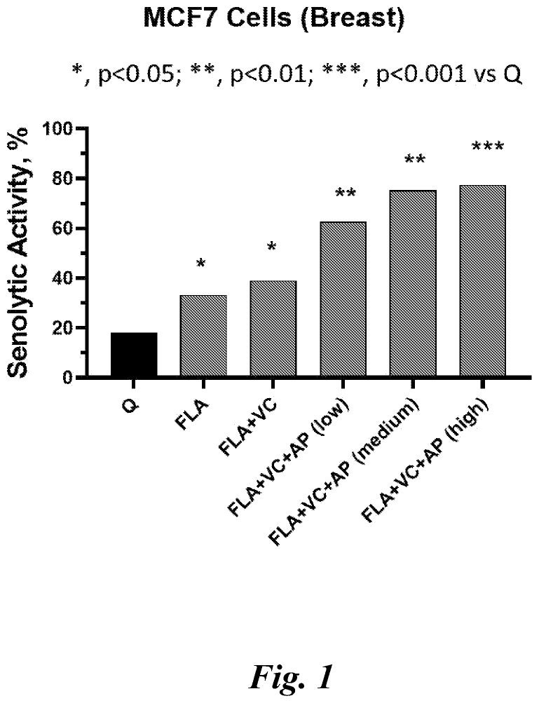

the FIG. 1 shows a first graph showing the results obtained with the substance according to the invention.

In the present document, the measurements, values, shapes and geometric references (such as perpendicularity and parallelism), when associated with words like “about” or other similar terms such as “approximately” or “substantially”, are to be considered as except for measurement errors or inaccuracies due to production and/or manufacturing errors, and, above all, except for a slight divergence from the value, measurements, shape, or geometric reference with which it is associated. For instance, these terms, if associated with a value, preferably indicate a divergence of not more than 10% of the value.

Moreover, when used, terms such as “first”, “second”, “higher”, “lower”, “main” and “secondary” do not necessarily identify an order, a priority of relationship or a relative position, but can simply be used to clearly distinguish between their different components.

The measurements and data reported in this text are to be considered, unless otherwise indicated, as carried out in the ICAO International Standard Atmosphere (ISO 2533).

The senotherapeutic substance according to the invention is for medical purposes for the treatment, preferably as selective as possible, of senescent cells.

The senotherapeutic substance preferably has a senolytic action and is therefore a senolytic food. The senolytic substance is used to eliminate, preferably selectively, senescent cells.

Alternatively, the senotherapeutic substance can have a senostatic action, that is, an action that blocks the senescence process.

The senotherapeutic substance can have, alternatively still, a senomorphic action, that is an action on the secretions of senescent cells.

The sinotherapeutics are therapeutic agents and methods that specifically target senescent cells, including their molecules and intracellular processes, and their released secretory substances. Senescent cells exhibit a unique and altered cell phenotype that arises in all tissues of an organism (including humans) as a consequence of many biological stressors. Among others, cellular senescence can be associated with aging and age-related diseases.

The sinotherapeutics can be further classified into at least two main categories:
Senolytics: agents that specifically eliminate senescent cells. Senolytics can eliminate senescent cells by inducing specific cell death mechanisms, including apoptosis, autophagy, necrosis, necroptosis or other forms of non-apoptotic programmed cell death (such as ferroptosis, pyroptosis, etc.). In some configurations, senolytics can target survival and anti-apoptotic pathways in senescent cells, known as senescent cell anti-apoptotic (SCAP) pathways.
Senomorphic: agents that specifically suppress the phenotype of senescent cells, without necessarily eliminating or killing senescent cells. Senomorphics modulate the functions and morphology of senescent cells, thus potentially delaying/preventing/inhibiting their formation, accumulation and pathological actions. In some configurations, the senomorphic includes inhibitors of the secretory associated senescence phenotype (SASP) and agents that specifically prevent cellular senescence.

The substance may have more specific advantages, and consequent uses, in the fields indicated in the list below and, more preferably, to treat disorders, which may be pathologies or aesthetic disorders or others preferably associated with senescence, indicated below with a terminology English scientific clear to the artisan of any language:
Idiopathic pulmonary fibrosis (IPF), chronic obstructive pulmonary disease (COPD), asthma, cystic fibrosis, emphysema, bronchiectasis, and age-related loss of pulmonary function;
Chronic kidney disease (CKD), interstitial nephritis, glomerulosclerosis/glomerulonephritis, acute kidney disease (AKD), kidney failure;
Liver fibrosis, chronic hepatitis, non-alcoholic fatty liver disease (NAFLD);
Pancreatic fibrosis, chronic pancreatitis;
Myocardial fibrosis, infarction;
Oral submucosa fibrosis;
Neurodegenerative Diseases, such as Alzheimer's, Parkinson's, Multiple Sclerosis, mild cognitive impairment, motor neuron dysfunction, Huntington's disease, dementia, etc.;
Neuropsychiatric disorders;
Toxicity or inflammation, induced by chemotherapies, radiotherapies, or any other medical procedure, such as for therapeutic, diagnostic, cosmetic purposes;
Acute and chronic viral diseases, such as HIV, Covid-19, etc.;
Osteoporosis, osteoarthritis, inflammatory bowel diseases (IBDs), inflammatory bowel syndrome (IBS), rheumatoid arthritis, oral mucositis, kyphosis, intervertebral disc degeneration, herniated intervertebral disc;
Adipose atrophy;
Sarcopenia, muscle/mobility loss due to aging, muscle fatigue;
Atherosclerosis, angina, arrhythmia, cardiomyopathy, cardiomyocyte hypertrophy, congestive heart failure, coronary artery disease, carotid artery disease, endocarditis, coronary thrombosis, myocardial infarction, hypertension, aortic aneurysm, cardiac diastolic dysfunction, hypercholesterolemia, hyperlipidemia, mitral valve prolapsed, peripheral vascular disease, cardiac stress resistance, cardiac fibrosis, brain aneurysm, and stroke;
Rare Diseases associated with aging and senescence, such as: aplastic anaemia, dyskeratosis congenita, Revetz syndrome, Hoyeraal-Hreidarsson syndrome, Lewy body dementia (LBD), amyloidosis, Paget's disease, diffuse idiopathic skeletal hyperostosis (DISH), multiple system atrophy (MSA), etc.;
Diabetes (Type 2, Type 1), diabetic ulcer, obesity, metabolic syndrome;
Wound healing;
Frailty;
Glaucoma, macular degeneration, cataracts, presbyopia, and vision loss;
Hearing Loss;
Immune function decline due to aging (Immunosenescence);
Alopecia, Hair Loss;
Melasma, discoloured skin, eczema, psoriasis, hyperpigmentation, nevi, rashes, atopic dermatitis, urticaria, diseases and disorders related to photosensitivity or photoaging, rhytides, pruritis, dysesthesia, eczematous eruptions, eosinophilic dermatosis, reactive neutrophilic dermatosis, pemphigoidus dermatosis, fibrohistocytic proliferations of skin, cutaneous lymphomas, and cutaneous lupus;
Diseases or pathological alterations or perfusion conditions associated to transplant of kidney, liver, lung, heart, pancreas or other organ, as well as of stem cells or other cells.

The said senotherapeutic substance can be used alone or, in combination with other known foods and drugs of the type selected from: senolytics, senomorphic, senostatic, senotherapeutic, cellular senescence promoters, and compounds that preserve the integrity of the tissues.

The senotherapeutic substance according to the invention preferably comprises flavonoids, fatty acids, and optionally phenolic acids and/or vitamins. The said components may be present together with other components or without other components.

The senotherapeutic substance preferably consists of a solid food or a liquid food, or again, alternatively, a substance or cream for topical use and an aeriform to be inhaled or other (for example, but not limited to, administered by injection).

Preferably the flavonoids are present as flavones, more preferably selected from one or more of quercetin, fisetin, apigenin and luteolin. Similar substances are for example marketed by Fluorochem Ltd. (UK), under the trademark of 047268 Quercetin.

In addition, flavonoids may include one or more of the following substances: rutin, isoquercetin, quercitrin, spireoside, hesperidin, hesperitin, diosmin, resveratrol, hydroxytyrosol, tyrosol, catechins, epicatechins, myricetin, epigallocatechin gallate, ellagic acid, curcumin, silystein, lutein, piperlongumine, iuglanin, loliolide, bromheolin, papain, allicin, lycopene, chemferol, naringin, sesamine, taxifolin, hyperoside.

The flavonoids are preferably present in quantities by weight comprised between 1 dg and 1 g, more preferably between 2 dg and 6 dg. These quantities preferably correspond to the daily quantity to be taken.

The fatty acids are preferably present in quantities by weight comprised between 1/50 and 25 times with respect to the quantity by weight of the flavonoids, more preferably said quantities are comprised between 1 and 10 times, more preferably between 3 and 8 times.

Preferably, the fatty acids are present in quantities ranging from 2 cg to 5 g, more preferably between 1 dg and 10 dg. These quantities preferably correspond to the daily quantity to be taken.

Preferably, the fatty acids are present as palmitic acid. Alternatively, or in addition, they can be selected from one or more of: acyl-ethanolaminides, oxylipins, lipoxins and their various technical formulations. Preferred, but not exhaustive, examples include: oleic acid, linolenic acid, conjugated linolenic acid, linoleic acid, conjugated linoleic acid, cannabinoid, palmitoylethanolamide (PEA), arachidonic acid, eicosapentaenoic acid, docosahexaenoic acid, docosanoic acid, lipoic acid. Similar substances are for example marketed by TCI Europe NV, under the trademark of P0002 Palmitic Acid.

The phenolic acids are preferably present in quantities by weight comprised between 1/100 and 4 times with respect to the quantity by weight of the flavonoids, more preferably said quantities are comprised between 30% and 100%, more preferably between 40% and 80%.

Preferably, the phenolic acids are present in quantities ranging from 1 cg to 8 dg, more preferably between 1 dg and 5 dg. These quantities preferably correspond to the daily quantity to be taken.

Preferably, the phenolic acids are present as gallic acid. Alternatively, or in addition, they can be selected from one or more of: vanillic acid, hydroxybenzoic acids, coumaric acid, ferulic acid, caffeic acid, hydroxycinnamic acids. Similar substances are for example marketed by TCI Europe NV, under the trademark of G0011 Gallic Acid Hydrate.

The vitamins are preferably present in quantities by weight comprised between 1/20 and 5 times with respect to the quantity by weight of the flavonoids, more preferably said quantities are comprised between 50% and 150%, more preferably still between 80% and 120%.

Preferably, the vitamins are present in quantities ranging from 1 cg to 1 g, more preferably between 5 cg and 5 dg. These quantities preferably correspond to the daily quantity to be taken.

Preferably, the vitamins are present as Vitamin C. Alternatively, or in addition, they can be chosen from one or more of: Vitamin D, Vitamin A, Vitamin B, Vitamin E. Similar substances are for example marketed by TCI Europe NV, under the trademark of A0537 L-Ascorbic Acid.

The disclosed senotherapeutic substance can be used alone or in combination with other supplements, topical creams, inhaled aeriforms, foods for special medical purposes, nutritional principles, essential minerals or known food antioxidants. Non-exhaustive examples of these are: carnosine, kinetine, GAL-duocarmicine, spermine, spermidine, zeaxanthin, digoxin, ouabain, avenanthramide C, urolithin, glucosamine, carnitine, bromelain, papain, fucoidan, zinc, lithium, manganese, magnesium, iron, calcium, selenium, chromium, phosphorus.

The described senotherapeutic substance can also be used alone or in combination with other known drugs of the type selected from: senolytics, senomorphic, senostatic, senotherapeutic, cellular senescence promoters, and compounds that preserve tissue integrity.

The invention therefore also defines a new process for eliminating senescent cells, or for solving problems associated with senescence by assuming the food or substance described and a new process for making substances or foods for curing the aforementioned problems.

Following experiments of the applicant, in which a senotherapeutic substance, including the quantities by weight of the various components indicated above, was administered to cells coming from patients, important senolytic activities were observed.

In particular, the senotherapeutic substance according to the realized invention, which showed marked senolytic activity in senescent epithelial cells of human breast, includes 3 flavonoids (fisetin, luteolin and apigenin), vitamin C and palmitic acid (FIG. 1). Briefly, senescence of MCF7 human mammary epithelium cells (human breast adenocarcinoma cells, ATCC® HTB-22™) was induced in vitro by treatment with doxorubicin (for 24 h), at a non-apoptotic concentration (200 nM), and subsequent cell recovery (in optimal culture medium, without treatments) for 10 days. The successful conversion to the senescent cell phenotype was confirmed by morphological changes (flattening and cell enlargement) and positivity for lysosomal beta-galactosidase (β-GAL). The SA-β-GAL (senescence-associated p-GAL) senescence assay was performed with the SA-β-Gal Staining Kit (Cell Signaling Technology, Inc., Danvers, MA). In this case, after fixation with 20% formaldehyde for 15 min at room temperature, the senescent cells were quantified by calculating the percentage of SA-β-GAL positive cells (colored blue) present in culture, examining ≥200 cells per well with the phase contrast microscope EVOS XL Cell Imaging System (Thermo Fisher; objective, 40×).

The senolytic activity (FIG. 1) on senescent MCF7 cells (in 96-well plates) was then evaluated after treatments (for 72 h) with the vehicle (DMSO; negative control), quercetin (Q, 5 μM; control positive), or the senotherapeutic substance according to the invention comprising the combination fisetin (F, 5 μM)+luteolin (L, 5 μM)+apigenin (A, 5 μM), alone or in the presence of palmitic acid (AP, used at 3 different concentrations: low, 10 μM; medium, 30 μM and high, 60 μM) and/or vitamin C (VC, 5 μM). At the end of the treatments, cell survival was quantified by crystal violet staining. In this case, the cells were fixed with paraformaldehyde (4%) in PBS for 20 min, washed (2× in distilled water) and stained with 1% crystal violet (for 20 min). After further washing with water (3×), 100 μl of acetic acid (10%) were added per well and the absorbance (λ=590 nm) measured with a Synergy spectrophotometer (AHSI). The experiment was carried out in quadruplicate and repeated three times, on three different days. Senolytic activity was expressed as % of senescent cells eliminated with respect to the control condition (DMSO). Results were presented as means±SEM (standard error of mean) and related analyzes were performed with Graph Pad Prism® 6.0 software (CA, USA).

Compared to quercetin, all the senotherapeutic combinations according to the invention examined exhibited significantly higher inhibitory activity on human MCF7 senescent cells (FIG. 1). In fact, the combination fisetin-luteolin-apigenin, alone or in the presence of vitamin C, has been shown to have a higher senolytic efficacy (≥100%) than that of quercetin, with significance (*, p<0.05) confirmed with the Student's t-test (FIG. 1). Of note, the addition of palmitic acid to the senotherapeutic combination further increased the senolytic efficacy of the substance according to the invention, with significantly higher dose-dependent synergistic effects than quercetin (**, p<0.01 and ***, p<0.001 with Student's t-test) and of maximum intensity (˜80% senolysis) at concentrations 30 μM (FIG. 1). The biological synergy of the chemical components constituting the senotherapeutic substance according to the invention described in these studies (fisetin+luteolin+apigenin+vitamin C+palmitic acid) is proven by the absence of significant senolytic effects on vitamin C and palmitic acid administered by alone (at the same doses investigated). These results therefore demonstrate that the senotherapeutic food has a specific, important and very promising senolytic activity, indicating that they could be developed as new senotherapeutic substances, and in particular senolytic, for humans.

1. A senotherapeutical substance comprising flavonoids and fatty acids.

2. The senotherapeutic substance according to claim 1, wherein said flavonoids are present in quantities by weight between 1 dg and 1 g.

3. The senotherapeutic substance according to claim 1, wherein said flavonoids are selected from one or more of quercetin, fisetin, apigenin and luteolin.

4. The senotherapeutic substance according to claim 1, wherein said fatty acids are present in quantities by weight ranging from 1 to 10 times with respect to the quantity by weight of said flavonoids.

5. The senotherapeutic substance according to claim 1, wherein said fatty acids are present as palmitic acid.

6. The senotherapeutic substance according to claim 1, further comprising phenolic acids.

7. The senotherapeutic substance according to claim 6, wherein said phenolic acids are present in quantities by weight ranging from 30% to 100% with respect to the quantity by weight of said flavonoids.

8. The senotherapeutic substance according to claim 4, wherein said phenolic acids are present as gallic acid.

9. The senotherapeutic substance according to claim 1, further comprising vitamins.

10. The senotherapeutic substance according to claim 1, consisting of a solid or liquid food.

11. The senotherapeutic substance according to claim 1, consisting of a substance or cream for topical use or an aeriform to be inhaled.

12. The therapeutic substance according to claim 1 for the treatment of one or more of the following disorders: Idiopathic pulmonary fibrosis (IPF), chronic obstructive pulmonary disease (COPD), asthma, cystic fibrosis, emphysema, bronchiectasis, and age-related loss of pulmonary function; Chronic kidney disease (CKD), interstitial nephritis, glomerulosclerosis/glomerulonephritis, acute kidney disease (AKD), kidney failure; Liver fibrosis, chronic hepatitis, non-alcoholic fatty liver disease (NASH); Pancreatic fibrosis, chronic pancreatitis; Myocardial fibrosis, infarction; Oral submucosa fibrosis; Neurodegenerative Diseases: Alzheimer's, Parkinson's, Multiple Sclerosis, mild cognitive impairment, motor neuron dysfunction, Huntington's disease, dementia; Neuropsychiatric disorders; Toxicity or inflammation, induced by chemotherapies, radiotherapies, or any other medical procedure, such as for therapeutic, diagnostic, cosmetic purposes; Acute and chronic viral diseases, such as HIV, Covid 19; Osteoporosis, osteoarthritis, inflammatory bowel diseases (IBDs), inflammatory bowel syndrome (IBS), rheumatoid arthritis, oral mucositis, kyphosis, intervertebral disc degeneration, herniated intervertebral disc; Adipose atrophy; Sarcopenia, muscle/mobility loss due to ageing, muscle fatigue; Atherosclerosis, angina, arrhythmia, cardiomyopathy, cardiomyocyte hypertrophy, congestive heart failure, coronary artery disease, carotid artery disease, endocarditis, coronary thrombosis, myocardial infarction, hypertension, aortic aneurysm, cardiac diastolic dysfunction, hypercholesterolemia, hyperlipidemia, mitral valve prolapsed, peripheral vascular disease, cardiac stress resistance, cardiac fibrosis, brain aneurysm, and stroke; Rare Diseases associated with ageing and senescence, such as: aplastic anaemia, dyskeratosis congenita, Revetz syndrome, Hoyeraal-Hreidarsson syndrome, Lewy body dementia (LBD), amyloidosis, Paget's disease, diffuse idiopathic skeletal hyperostosis (DISH), multiple system atrophy (MSA); Diabetes (Type 2, Type 1), diabetic ulcer, obesity, metabolic syndrome; Wound healing; Frailty; Glaucoma, macular degeneration, cataracts, presbyopia, and vision loss; Hearing Loss; Immune function decline due to ageing (Immunosenescence); Alopecia, Hair Loss; Melasma, discoloured skin, eczema, psoriasis, hyperpigmentation, nevi, rashes, atopic dermatitis, urticaria, diseases and disorders related to photosensitivity or photoaging, rhytides, pruritis, dysesthesia, eczematous eruptions, eosinophilic dermatosis, reactive neutrophilic dermatosis, pemphigus, pemphigoid, immunobullous dermatosis, fibrohistocytic proliferations of skin, cutaneous lymphomas, and cutaneous lupus; Diseases or pathological alterations or perfusion conditions associated to transplant of kidney, liver, lung, heart, pancreas or other organ, as well as of stem cells or other cells.

13. A method of manufacturing a medicament for senotherapeutic use comprising adding to the medicament a substance according to claim 1.

14. A method of use of the substance according to claim 1, comprising administering the substance to a person for eliminating senescent cells, or suppressing the phenotype of senescent cells, for the treatment of one or more of the following disorders: Idiopathic pulmonary fibrosis (IPF), chronic obstructive pulmonary disease (COPD), asthma, cystic fibrosis, emphysema, bronchiectasis, and age-related loss of pulmonary function; Chronic kidney disease (CKD), interstitial nephritis, glomerulosclerosis/glomerulonephritis, acute kidney disease (AKD), kidney failure; Liver fibrosis, chronic hepatitis, non-alcoholic fatty liver disease (NAFLD); Pancreatic fibrosis, chronic pancreatitis; Myocardial fibrosis, infarction; Oral submucosa fibrosis; Neurodegenerative Diseases: Alzheimer's, Parkinson's, Multiple Sclerosis, mild cognitive impairment, motor neuron dysfunction, Huntington's disease, dementia; Neuropsychiatric disorders; Toxicity or inflammation, induced by chemotherapies, radiotherapies, HIV, stem cell transplant, other drugs; Osteoporosis, osteoarthritis, inflammatory bowel diseases (IBDs), inflammatory bowel syndrome (IBS), rheumatoid arthritis, oral mucositis, kyphosis, intervertebral disc degeneration, herniated intervertebral disc; Adipose atrophy; Sarcopenia, muscle/mobility loss due to aging, muscle fatigue; Atherosclerosis, angina, arrhythmia, cardiomyopathy, cardiomyocyte hypertrophy, congestive heart failure, coronary artery disease, carotid artery disease, endocarditis, coronary thrombosis, myocardial infarction, hypertension, aortic aneurysm, cardiac diastolic dysfunction, hypercholesterolemia, mitral valve prolapse, peripheral vascular disease, cardiac stress resistance, cardiac fibrosis, brain aneurysm, and stroke; Rare Diseases associated with aging and senescence, such as: aplastic anaemia, dyskeratosis congenita, Revetz syndrome, Hoyeraal-Hreidarsson syndrome, Lewy body dementia (LBD), amyloidosis, Paget's disease, diffuse idiopathic skeletal hyperostosis (DISH), multiple system atrophy (MSA); Diabetes (Type 2, Type 1), diabetic ulcer, obesity, metabolic syndrome; Wound healing; Frailty; Glaucoma, macular degeneration, cataracts, presbyopia, and vision loss; Hearing Loss; Immune function decline due to aging (Immunosenescence); Alopecia, Hair Loss; Melasma, discoloured skin, eczema, psoriasis, hyperpigmentation, nevi, rashes, atopic dermatitis, urticaria, diseases and disorders related to photosensitivity or photoaging, rhytides, pruritis, dysesthesia, eczematous eruptions, eosinophilic dermatosis, reactive neutrophilic dermatosis, pemphigoidus dermatosis, fibrohistocytic proliferations of skin, cutaneous lymphomas, and cutaneous lupus; Transplant of kidney, liver, lung, heart, pancreas or other organ.

15. A method of use of the substance according to claim 1, comprising administering the substance in combination with other known and/or drugs of the type selected from: senolytics, senomorphic, senostatic, senotherapeutic, cellular senescence promoters, and compounds that preserve tissue integrity, to a person for eliminating senescent cells, or suppressing the phenotype of senescent cells, for treatment of one or more of the following disorders: Idiopathic pulmonary fibrosis (IPF), chronic obstructive pulmonary disease (COPD), asthma, cystic fibrosis, emphysema, bronchiectasis, and age-related loss of pulmonary function; Chronic kidney disease (CKD), interstitial nephritis, glomerulosclerosis/glomerulonephritis, acute kidney disease (AKD), kidney failure; Liver fibrosis, chronic hepatitis, non-alcoholic fatty liver disease (NAFLD); Pancreatic fibrosis, chronic pancreatitis; Myocardial fibrosis, infarction; Oral submucosa fibrosis; Neurodegenerative Diseases: Alzheimer's, Parkinson's, Multiple Sclerosis, mild cognitive impairment, motor neuron dysfunction, Huntington's disease, dementia; Neuropsychiatric disorders; Toxicity or inflammation, induced by chemotherapies, radiotherapies, HIV, stem cell transplant, other drugs; Osteoporosis, osteoarthritis, inflammatory bowel diseases (IBDs), inflammatory bowel syndrome (IBS), rheumatoid arthritis, oral mucositis, kyphosis, intervertebral disc degeneration, herniated intervertebral disc; Adipose atrophy; Sarcopenia, muscle/mobility loss due to aging, muscle fatigue; Atherosclerosis, angina, arrhythmia, cardiomyopathy, cardiomyocyte hypertrophy, congestive heart failure, coronary artery disease, carotid artery disease, endocarditis, coronary thrombosis, myocardial infarction, hypertension, aortic aneurysm, cardiac diastolic dysfunction, hypercholesterolemia, mitral valve prolapse, peripheral vascular disease, cardiac stress resistance, cardiac fibrosis, brain aneurysm, and stroke; Rare Diseases associated with aging and senescence, such as: aplastic anaemia, dyskeratosis congenita, Revetz syndrome, Hoyeraal-Hreidarsson syndrome, Lewy body dementia (LBD), amyloidosis, Paget's disease, diffuse idiopathic skeletal hyperostosis (DISH), multiple system atrophy (MSA); Diabetes (Type 2, Type 1), diabetic ulcer, obesity, metabolic syndrome; Wound healing; Frailty; Glaucoma, macular degeneration, cataracts, presbyopia, and vision loss; Hearing Loss; Immune function decline due to aging (Immunosenescence); Alopecia, Hair Loss; Melasma, discoloured skin, eczema, psoriasis, hyperpigmentation, nevi, rashes, atopic dermatitis, urticaria, diseases and disorders related to photosensitivity or photoaging, rhytides, pruritis, dysesthesia, eczematous eruptions, eosinophilic dermatosis, reactive neutrophilic dermatosis, pemphigoidus dermatosis, fibrohistocytic proliferations of skin, cutaneous lymphomas, and cutaneous lupus; Transplant of kidney, liver, lung, heart, pancreas or other organ.

16. A composition comprising the substance according to claim 1, for eliminating senescent cells, or suppressing the phenotype of senescent cells, for treatment of one or more of the following disorders: Idiopathic pulmonary fibrosis (IPF), chronic obstructive pulmonary disease (COPD), asthma, cystic fibrosis, emphysema, bronchiectasis, and age-related loss of pulmonary function; Chronic kidney disease (CKD), interstitial nephritis, glomerulosclerosis/glomerulonephritis, acute kidney disease (AKD), kidney failure; Liver fibrosis, chronic hepatitis, non-alcoholic fatty liver disease (NAFLD); Pancreatic fibrosis, chronic pancreatitis; Myocardial fibrosis, infarction; Oral submucosa fibrosis; Neurodegenerative Diseases: Alzheimer's, Parkinson's, Multiple Sclerosis, mild cognitive impairment, motor neuron dysfunction, Huntington's disease, dementia; Neuropsychiatric disorders; Toxicity or inflammation, induced by chemotherapies, radiotherapies, HIV, stem cell transplant, other drugs; Osteoporosis, osteoarthritis, inflammatory bowel diseases (IBDs), inflammatory bowel syndrome (IBS), rheumatoid arthritis, oral mucositis, kyphosis, intervertebral disc degeneration, herniated intervertebral disc; Adipose atrophy; Sarcopenia, muscle/mobility loss due to aging, muscle fatigue; Atherosclerosis, angina, arrhythmia, cardiomyopathy, cardiomyocyte hypertrophy, congestive heart failure, coronary artery disease, carotid artery disease, endocarditis, coronary thrombosis, myocardial infarction, hypertension, aortic aneurysm, cardiac diastolic dysfunction, hypercholesterolemia, mitral valve prolapse, peripheral vascular disease, cardiac stress resistance, cardiac fibrosis, brain aneurysm, and stroke; Rare Diseases associated with aging and senescence, such as: aplastic anaemia, dyskeratosis congenita, Revetz syndrome, Hoyeraal-Hreidarsson syndrome, Lewy body dementia (LBD), amyloidosis, Paget's disease, diffuse idiopathic skeletal hyperostosis (DISH), multiple system atrophy (MSA); Diabetes (Type 2, Type 1), diabetic ulcer, obesity, metabolic syndrome; Wound healing; Frailty; Glaucoma, macular degeneration, cataracts, presbyopia, and vision loss; Hearing Loss; Immune function decline due to aging (Immunosenescence); Alopecia, Hair Loss; Melasma, discoloured skin, eczema, psoriasis, hyperpigmentation, nevi, rashes, atopic dermatitis, urticaria, diseases and disorders related to photosensitivity or photoaging, rhytides, pruritis, dysesthesia, eczematous eruptions, eosinophilic dermatosis, reactive neutrophilic dermatosis, pemphigoidus dermatosis, fibrohistocytic proliferations of skin, cutaneous lymphomas, and cutaneous lupus; Transplant of kidney, liver, lung, heart, pancreas or other organ.

17. A composition comprising the substance according to claim 1 in combination with other known foods and/or drugs of the type selected from: senolytics, senomorphic, senostatic, senotherapeutic, cellular senescence promoters, and compounds that preserve tissue integrity, for eliminating senescent cells, or suppressing the phenotype of senescent cells, for treatment of one or more of the following disorders: Idiopathic pulmonary fibrosis (IPF), chronic obstructive pulmonary disease (COPD), asthma, cystic fibrosis, emphysema, bronchiectasis, and age-related loss of pulmonary function; Chronic kidney disease (CKD), interstitial nephritis, glomerulosclerosis/glomerulonephritis, acute kidney disease (AKD), kidney failure; Liver fibrosis, chronic hepatitis, non-alcoholic fatty liver disease (NAFLD); Pancreatic fibrosis, chronic pancreatitis; Myocardial fibrosis, infarction; Oral submucosa fibrosis; Neurodegenerative Diseases: Alzheimer's, Parkinson's, Multiple Sclerosis, mild cognitive impairment, motor neuron dysfunction, Huntington's disease, dementia; Neuropsychiatric disorders; Toxicity or inflammation, induced by chemotherapies, radiotherapies, HIV, stem cell transplant, other drugs; Osteoporosis, osteoarthritis, inflammatory bowel diseases (IBDs), inflammatory bowel syndrome (IBS), rheumatoid arthritis, oral mucositis, kyphosis, intervertebral disc degeneration, herniated intervertebral disc; Adipose atrophy; Sarcopenia, muscle/mobility loss due to aging, muscle fatigue; Atherosclerosis, angina, arrhythmia, cardiomyopathy, cardiomyocyte hypertrophy, congestive heart failure, coronary artery disease, carotid artery disease, endocarditis, coronary thrombosis, myocardial infarction, hypertension, aortic aneurysm, cardiac diastolic dysfunction, hypercholesterolemia, mitral valve prolapse, peripheral vascular disease, cardiac stress resistance, cardiac fibrosis, brain aneurysm, and stroke; Rare Diseases associated with aging and senescence, such as: aplastic anaemia, dyskeratosis congenita, Revetz syndrome, Hoyeraal-Hreidarsson syndrome, Lewy body dementia (LBD), amyloidosis, Paget's disease, diffuse idiopathic skeletal hyperostosis (DISH), multiple system atrophy (MSA); Diabetes (Type 2, Type 1), diabetic ulcer, obesity, metabolic syndrome; Wound healing; Frailty; Glaucoma, macular degeneration, cataracts, presbyopia, and vision loss; Hearing Loss; Immune function decline due to aging (Immunosenescence); Alopecia, Hair Loss; Melasma, discoloured skin, eczema, psoriasis, hyperpigmentation, nevi, rashes, atopic dermatitis, urticaria, diseases and disorders related to photosensitivity or photoaging, rhytides, pruritis, dysesthesia, eczematous eruptions, eosinophilic dermatosis, reactive neutrophilic dermatosis, pemphigoidus dermatosis, fibrohistocytic proliferations of skin, cutaneous lymphomas, and cutaneous lupus; Transplant of kidney, liver, lung, heart, pancreas or other organ.
